(19) United States
(12) Patent Application Publication
(10) Pub. No.: US 2012/0244201 A1
(43) Pub. Date: Sep. 27, 2012

(54) PROCESS FOR THE PREPARATION OF AROMATIZED CHEWING FOAMS FOR COSMETIC PRODUCTS

(75) Inventors: Steffen HOFACKER, Odenthal (DE); Meike Niesten, Koln (DE); Thorsten Rische, Unna (DE); Sebastian Dörr, Duesseldorf (DE); Thorsten Krämer, Langenfeld (DE); Dirk Schwannecke, Murray Hill, NJ (US); Gabriele Rohé, Holzminden (DE); Jens Hepperle, Koln (DE); Hartwig Kempkes, Engelskirchen (DE); Claudia Letmathe, Bergisch Gladbach (DE)

(73) Assignee: Bayer MaterialScience AG Patents & Licensing, Leverkusen (DE)

(21) Appl. No.: 13/489,821

(22) Filed: Jun. 6, 2012

Related U.S. Application Data
(63) Continuation of application No. 12/253,559, filed on Oct. 17, 2008.
(60) Provisional application No. 60/999,611, filed on Oct. 19, 2007.

(51) Int. Cl. A61Q 11/00; A61K 9/68; A61K 8/02

(52) U.S. Cl. 424/401; 427/2.29

(57) ABSTRACT

The invention relates to a process for the preparation of novel aromatized chewing foams for the oral care sector based on polyurethane-polyureas and aromatizing compositions.

### RELATED APPLICATIONS

This application is a Continuation of U.S. Ser. No. 12/253,559, which was filed Oct. 17, 2008, which claims priority to U.S. Provisional Application No. 60/999,611, filed Oct. 19, 2007, each of which is incorporated herein by reference in their entireties.

### BACKGROUND OF THE INVENTION

The invention relates to a process for the preparation of novel aromatized chewing foams for the oral care sector based on polyurethane-polyureas and aromatizing compositions.

Organic polymers are widespread as raw materials in cosmetic products. They may be found in all sorts of cosmetic products such as, for example, hair sprays, hair gels, mascara, lipsticks, creams etc. In the oral care sector, polymers may be found, for example, in the form of toothbrushes, dental flosses etc.

On account of the increasing need of society for oral care for the periods between meal times or following consumption, for example, of a between-meal snack or other products consumed for pleasure (such as, for example, sweets, nicotine, alcohol, etc.) or else on account of increased mobility (for example during air or train travel) in which conventional teeth cleaning with water, toothpaste and toothbrush is not possible, in the past products such as dental care chewing gums and also dental care wipes have been developed.

Dental care chewing gums consist essentially of so-called chewing gum base. This in turn consists of natural or synthetic polymers such as, for example, latex, polyvinyl ethers, polyisobutylene vinyl ether, polyisobutene, etc. Such dental care chewing gums generally comprise, as dental care agents, pH-controlling substances which thus counteract the development of tooth decay (caries). On account of their plastic behaviour, such dental care chewing gums, however, barely contribute to cleaning the chewing surfaces or tooth sides. In addition, chewing gums generally have the disadvantage that they often have to be mechanically removed from public streets and areas, and be disposed of, which leads to considerable cleaning expenditure—on account of their adhesive properties—of the floor and road surfaces.

Dental care wipes (for example Oral-B Brush Aways™, Gillette GmbH & Co. OHG, Germany) are characterized in that they achieve a good cleaning effect of the tooth sides by attaching the dental care wipe to a finger and by rubbing the teeth. However, the mode of using such dental cleansing wipes in public has gained little acceptance for aesthetic reasons and is thus no alternative to using a conventional toothbrush.

DE 102006019742.9 describes novel chewing foams based on polyurethane-polyureas. It has now been found that, for producing such aromatized chewing foams, a special process is required in order to ensure the quality of these chewing foams.

### EMBODIMENTS OF THE INVENTION

An embodiment of the present invention is a process for preparing aromatized chewing foams, comprising
i) formulating one or more polyurethane-polyurea dispersions (I) with foam auxiliaries (III), optionally with thickeners (IV), and optionally with cosmetic additives (V);
ii) foaming the formulation of i);
iii) applying the foamed formulation of ii) to a substrate;
iv) drying the foamed formulation applied to the substrate of iii);
v) shaping the substrate of iv);
vi) refining the surface of the substrate of v);
with the proviso that aromatizing compositions (II) are added in at least one of i) to vi).

Another embodiment of the present invention is the above process, wherein said polyurethane-polyurea dispersions (I) obtained by
A) preparing isocyanate-functional prepolymers of
   a1) aliphatic or cycloaliphatic polyisocyanates;
   a2) polymeric polyols with number-average molecular weights of from 400 to 8000 g/mol and OH functionalities of from 1.5 to 6;
   a3) optionally hydroxy-functional, ionic or potentially ionic and/or nonionic hydrophilizing agents;
B) completely or partially reacting the free NCO groups of said isocyanate-functional prepolymers with
   b1) amino-functional compounds with molecular weights of from 32 to 400 g/mol; and/or
   b2) amino-functional, ionic, or potentially ionic hydrophilizing agents;
   with chain extension, and dispersing the prepolymers in water before, during or after B), wherein any potentially ionic groups present may be converted to the ionic form by partial or complete reaction with a neutralizing agent.

Another embodiment of the present invention is the above process, wherein a1) is selected from the group consisting of 1,6-hexamethylene diisocyanate, isophorone diisocyanate, the isomeric bis(4,4′-isocyanatocyclohexyl)methanes, or mixtures thereof, and a2) is at least 70% by weight of a mixture of polycarbonate polyols and polytetramethylene glycol polyols, based on the total weight of the components a2).

Another embodiment of the present invention is the above process, wherein said aromatizing compositions (II) comprise sensorily effective substances which are volatile and are perceptible orthonasally and/or retronasally (aroma substances), or are nonvolatile and are perceptible through interaction with the taste receptors of the human tongue (taste substances).

Another embodiment of the present invention is the above process, wherein said aroma substances comprise a combination of refreshing and cooling active ingredients as.

Another embodiment of the present invention is the above process, wherein said taste substances comprise sugar substitutes, sweeteners and/or substances which have a pungent taste, stimulate the flow of saliva in the mouth, cause a feeling of heat and/or a tingling feeling on the skin or on the mucosa.

Another embodiment of the present invention is the above process, wherein said sensorily effective substances are incorporated into a matrix as carrier substance.

Another embodiment of the present invention is the above process, wherein foam auxiliaries (III), thickeners (IV) and cosmetic additives (V) are formulated with said one or more polyurethane-polyurea dispersions.

Another embodiment of the present invention is the above process, wherein said foam auxiliaries are selected from the group consisting of sodium lauryl sulphate, alkyl polyglycoside sulphosuccinamides, ammonium stearate, or mixtures thereof.

Another embodiment of the present invention is the above process, wherein no cariogenic substances during the preparation of said aromatized chewing foams and said aromatized chewing foams do not exceed the critical value of pH 5.7 when carrying out an in vivo plaque pH test.

Another embodiment of the present invention is the above process, wherein the drying in iv) is achieved with microwave radiation at a power of from 250 to 6000 W per kilogram of the foam to be dried.

Another embodiment of the present invention is the above process, wherein the drying in iv) is achieved with conventional thermal drying in addition to said microwave radiation.

Another embodiment of the present invention is the above process, wherein an aromatizing composition (II) is added in i) and further comprising applying a further aromatizing composition (II) as a coating (VII) in vi) to the surface of the ready-shaped and dried chewing foam and subsequently drying said coating (VII).

Another embodiment of the present invention is the above process, wherein an aromatizing composition (II) is added in i) and further comprising applying a further aromatizing composition (II) in the form of an aqueous slurry as coating (VII) in vi) to the surface of the ready-shaped and dried chewing foam and subsequently drying said coating (VII).

Another embodiment of the present invention is the above process, further comprising applying a coating (VI) after iv) and before applying the aroma coating (VII).

Yet another embodiment of the present invention is an aromatized chewing foam obtained by the above process.

### DESCRIPTION OF THE INVENTION

It has now been found that such chewing foams can be prepared particularly advantageously by a special process.

The present invention provides a process for the preparation of such aromatized chewing foams, in which
i) one or more polyurethane-polyurea dispersions (I) are formulated with foam auxiliaries (III), optionally with thickeners (IV) and optionally with cosmetic additives (V),
ii) these compositions are then foamed
iii) applied to a substrate,
iv) dried and
v) subjected to shaping, where finally
vi) surface refining can take place,
with the proviso that, in at least one of the steps i) to vi) passed through, aromatizing compositions (II) are added.

Such polyurethane-polyurea dispersions (I) used in i) are obtainable by preparing
A) isocyanate-functional prepolymers of
   a1) aliphatic or cycloaliphatic polyisocyanates
   a2) polymeric polyols with number-average molecular weights of from 400 to 8000 g/mol and OH functionalities of from 1.5 to 6,
   a3) optionally hydroxy-functional, ionic or potentially ionic and/or nonionic hydrophilizing agents,
B) then completely or partially reacting their free NCO groups with
   b1) amino-functional compounds with molecular weights of from 32 to 400 g/mol and/or
   b2) amino-functional, ionic or potentially ionic hydrophilizing agents
      with chain extension, and dispersing the prepolymers in water before, during or after step B), where any potentially ionic groups present may be converted to the ionic form by partial or complete reaction with a neutralizing agent.

Isocyanate-reactive groups are, for example, amino groups, hydroxy groups or thiol groups.

In a1), 1,6-hexamethylene diisocyanate, isophorone diisocyanate, the isomeric bis(4,4′-iso-cyanatocyclohexyl)methanes, and mixture thereof are typically used.

The use of modified diisocyanates having a uretdione, isocyanurate, urethane, allophanate, biuret, iminooxadiazinedione and/or oxadiazinetrione structure, and also the nonmodified polyisocyanates having more than 2 NCO groups per molecule, such as 4-isocyanatomethyl-1,8-octane diisocyanate (nonan triisocyanate) or triphenylmethane 4,4′,4″-triisocyanate, is likewise possible.

The compounds of component a1) are particularly preferably polyisocyanates or polyisocyanate mixtures of the abovementioned type having exclusively aliphatically and/or cycloaliphatically bonded isocyanate groups and an average NCO functionality of the mixture of from 2 to 4, preferably 2 to 2.6 and particularly preferably 2 to 2.4.

As components in a2), polymeric polyols with number-average molecular weights of from 400 to 6000 g/mol, particularly preferably from 600 to 3000 g/mol, are used. These preferably have OH functionalities of from 1.8 to 3, particularly preferably from 1.9 to 2.1.

Such polymeric polyols which are known per se in polyurethane coating technology are polyester polyols, polycarbonate polyols, polyether polyols, polyacrylate polyols, polyester polycarbonate polyols and polyether carbonate polyols. These can be used in a2) individually or in any desired mixtures with one another.

The polymeric polyols of the abovementioned type used are preferably those having an aliphatic backbone. Preference is given to using aliphatic polycarbonate polyols, polyether polyols or any desired mixtures thereof.

Preferred embodiments of the polyurethane dispersions (I) comprise, as component a2), a mixture of polycarbonate polyols and polytetramethylene glycol polyols, where the fraction in the mixture is from 35 to 70% by weight of polytetramethylene glycol polyols and 30 to 65% by weight of polycarbonate polyols, with the proviso that the sum of the weight percentages of the polycarbonate and polytetramethylene glycol polyols is 100% by weight.

Hydroxy-functional, ionic or potentially ionic hydrophilizing agents a3) are understood as meaning all compounds which have at least one isocyanate-reactive hydroxy group and at least one functionality, such as, for example, —COOY, —SO₃Y, —PO(OY)₂ (Y for example ═H, NH₄, metal cation), —NR₂, —NR₃ (R═H, alkyl, aryl), which, on interaction with aqueous media, enters into a pH-dependent dissociation equilibrium and, in this way, may be negatively, positively or neutrally charged.

Suitable ionically or potentially ionically hydrophilizing compounds corresponding to the definition of component a3) are, for example, mono- and dihydroxycarboxylic acids, mono- and dihydroxysulphonic acids, and also mono- and dihydroxyphosphonic acids and their salts, such as dimethylolpropionic acid, dimethylbutyric acid, hydroxypivalic acid, malic acid, citric acid, glycolic acid, lactic acid, the propoxylated adduct of 2-butenediol and NaHSO₃, for example described in DE-A 2 446 440 (pages 5-9, formula I-III), and compounds which contain, as hydrophilic structural components, for example amine-based building blocks such as N-methyldiethanolamine which can be converted into cationic groups.

Preferred ionic or potentially ionic hydrophilizing agents of component a3) are those of the abovementioned type which have an anionically hydrophilizing effect, preferably via carboxy or carboxylate and/or sulphonate groups.

Particularly preferred ionic or potentially ionic hydrophilizing agents are those which contain carboxyl and/or sulphonate groups as anionic or potentially anionic groups, such as the salts of dimethylolpropionic acid or dimethylolbutyric acid.

Suitable nonionically hydrophilizing compounds of component a3) are, for example, polyoxyalkylene ethers which contain at least one hydroxy group as isocyanate-reactive group.

Examples are the monohydroxy-functional polyalkylene oxide polyether alcohols having a statistical average of from 5 to 70, preferably 7 to 55, ethylene oxide units per molecule, as are accessible in a manner known per se by alkoxylating suitable starter molecules (e.g. in Ullmanns Encyclopadie der technischen Chemie, 4th edition, volume 19, Verlag Chemie, Weinhcim, pp. 31-38).

These are either pure polyethylene oxide ethers or mixed polyalkylene oxide ethers, in which case they comprise at least 30 mol %, preferably at least 40 mol %, ethylene oxide units, based on all alkylene oxide units present.

Particularly preferred nonionic compounds are mono functional mixed polyalkylene oxide polyethers which have 40 to 100 mol % ethylene oxide units and 0 to 60 mol % propylene oxide units.

Suitable starter molecules for such nonionic hydrophilizing agents are saturated monoalcohols, such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as, for example, diethylene glycol monobutyl ether, unsaturated alcohols, such as allyl alcohol, 1,1-dimethylallyl alcohol or oleyl alcohol, aromatic alcohols, such as phenol, the isomeric cresols or methoxyphenols, araliphatic alcohols, such as benzyl alcohol, anisyl alcohol or cinnamyl alcohol, secondary monoamines, such as dimethylamine, diethylamine, dipropylamine, diisopropylamine, dibutylamine, bis(2-ethylhexyl)-amine, N-methyl- and N-ethylcyclohexylamine or dicyclohexylamine, and heterocyclic secondary amines, such as morpholine, pyrrolidine, piperidine or 1H-pyrazole. Preferred starter molecules are saturated monoalcohols of the abovementioned type. Particular preference is given to using diethylene glycol monobutyl ether or n-butanol as starter molecules.

Alkylene oxides suitable for the alkoxylation reaction are, in particular, ethylene oxide and propylene oxide, which can be used in the alkoxylation reaction in any desired sequence or else in a mixture.

As component b1) it is possible to use di- or polyamines, such as 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, and 4,4-diaminodicyclohexylmethane and/or dimethylethylenediamine.

Moreover, compounds which, besides a primary amino group, also have secondary amino groups or, besides an amino group (primary or secondary), also have OH groups, can also be used as component b1). Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines, such as N-amino ethyl ethanol amine, ethanolamine, 3-aminopropanol, neopentanolamine.

In addition, as component b1) it is also possible to use mono functional amine compounds, such as, for example, methylamine, ethylamine, propylamine, butylamine, octylamine, laurylamine, stearylamine, isononyloxypropylamine, dimethyl amine, diethylamine, dipropylamine, dibutylamine, N-methylaminopropylamine, diethyl(methyl)aminopropylamine, morpholine, piperidine, or suitable substituted derivatives thereof, amidamines of diprimary amines and monocarboxylic acids, monoketimines of diprimary amines, primary/tertiary amines, such as N,N-dimethylaminopropylamine.

Preference is given to using 1,2-ethylenediamine, 1,4-diaminobutane, isophoronediamine and diethylenetriamine.

Ionically or potentially ionically hydrophilizing compounds of component b2) are understood as meaning all compounds which have at least one isocyanate-reactive amino group and also at least one functionality, such as, for example, —COOY, —SO₃Y, —PO(OY)₂ (Y for example ═H, NH₄, metal cation), which, upon interaction with aqueous media, enters into a pH-dependent dissociation equilibrium and, in this way, may be positively, negatively or neutrally charged.

Suitable ionically or potentially ionically hydrophilizing compounds are, for example, mono- and diaminocarboxylic acids, mono- and diaminosulphonic acids and mono- and diaminophosphonic acids and their salts. Examples of such ionic or potentially ionic hydrophilizing agents are N-(2-aminoethyl)-13-alanine, 2-(2-aminoethylamino)ethanesulphonic acid, ethylenediaminepropylsulphonic or -butylsulphonic acid, 1,2- or 1,3-propylenediamine-β-ethylsulphonic acid, glycine, alanine, taurine, lysine, 3,5-diaminobenzoic acid and the addition product of IPDI and acrylic acid (EP-A 0 916 647, Example 1). In addition, it is also possible to use cyclohexylamino-propanesulphonic acid (CAPS) from WO-A 01/88006 as anionic or potentially anionic hydrophilizing agent.

Preferred ionic or potentially ionic hydrophilizing agents b2) are those which contain carboxyl and/or sulphonate groups as anionic or potentially anionic groups, such as the salts of N-(2-aminoethyl)-β-alanine, of 2-(2-amino ethylamino)ethanesulphonic acid or of the addition product of IPDI and acrylic acid (EP-A 0 916 647, Example 1).

For the hydrophilization, preference is given to using a mixture of anionic or potentially anionic hydrophilizing agents and nonionic hydrophilizing agents.

The ratio of NCO groups of the compounds of component a1) to NCO-reactive groups of the components a2) to a3) during the preparation of the NCO-functional prepolymer is 1.2 to 3.0, preferably 1.3 to 2.5.

The amino-functional compounds in stage B) are used in an amount such that the equivalent ratio of isocyanate-reactive amino groups of these compounds to the free isocyanate groups of the prepolymer is 50 to 125%, preferably between 60 and 120%.

In a preferred embodiment, use is made of anionically and nonionically hydrophilized polyurethane dispersions, where, for their preparation, the components a1) to a3) and b1) to b2) are used in the following amounts, the individual amounts preferably adding up to 100% by weight:

10 to 30% by weight of component a1),
65 to 85% by weight of a2),
0.5 to 14% by weight sum of component b1)
0.1 to 13.5% by weight sum of components a3) and b2), where, based on the total amounts of the components a1) to a3), 0.5 to 3.0% by weight of anionic or potentially anionic hydrophilizing agents are used.

Particularly preferred embodiments of the polyurethane dispersions (I) comprise, as component a1), isophorone diisocyanate and/or 1,6-hexamethylene diisocyanate and/or the isomeric bis(4,4′-isocyanatocyclohexyl)methanes in combination with a2) a mixture of polycarbonate polyols and polytetramethylene glycol polyols.

The respective fraction of the polymeric polyols in the mixture a2) is 35 to 70% by weight of polytetramethylene glycol polyols and 30 to 65% by weight of polycarbonate polyols, in each case with the proviso that the sum of the percentages by weight of the polycarbonate polyols and polytetramethylene glycol polyols is 100% by weight.

The preparation of such polyurethane dispersions can be carried out in one or more stage(s) in homogeneous or in multistage reaction, partially in disperse phase. Complete or partial polyaddition of a1) to a3) is followed by a dispersion, emulsification or dissolution step. Subsequently, if appropriate, a further polyaddition or modification in disperse phase takes place.

In this connection, it is possible to use all processes known from the prior art, such as, for example, the prepolymer mixing method, acetone method or melt dispersion method. Preferably, the process proceeds via the acetone method.

For the preparation according to the acetone method, the constituents a2) to a3), which must not have any primary or secondary amino groups, and the polyisocyanate component a1) for the preparation of an isocyanate-functional polyurethane prepolymer are customarily initially introduced in whole or in part and, if appropriate, diluted with a solvent that is miscible with water but inert towards isocyanate groups, and heated to temperatures in the range from 50 to 120° C. To accelerate the isocyanate addition reaction, the catalysts known in polyurethane chemistry are used.

Suitable solvents are the customary aliphatic, keto-functional solvents, such as acetone, 2-butanone, which can be added not only at the start of the preparation, but also, if appropriate, in parts later on. Preference is given to acetone and 2-butanone.

Subsequently, any constituents of a1) to a3) not yet added at the start of the reaction are metered in.

The reaction of the components a1) to a3) to give the prepolymer takes place partially or completely, but preferably completely. This thus gives polyurethane prepolymers which contain free isocyanate groups, without a diluent or in solution.

Subsequently, in a further process step, if this has not yet occurred, or has occurred only partially, the resulting prepolymer is dissolved with the help of aliphatic ketones, such as acetone or 2-butanone.

The aminic components b1) and b2) can, if appropriate, be used in water-diluted or solvent-diluted form in the process according to the invention individually or in mixtures, where, in principle, any sequence of addition is possible.

If water or organic solvents are co-used as diluents, then the diluent content in the component used in B) for chain extension is preferably 30 to 95% by weight.

Dispersion preferably takes place after the chain extension. For this, either the dissolved and chain-extended polyurethane polymer is introduced, optionally with severe shear, such as, for example, vigorous stirring, into the dispersion water or, vice versa, the dispersion water is stirred into the chain-extended polyurethane polymer solutions. Preferably, the water is added to the dissolved chain-extended polyurethane polymer.

The solvent still present in the dispersions after the dispersion step is usually then removed by distillation. Removal as early as during the dispersion is likewise possible.

The residual content of organic solvents in the dispersions essential to the invention is typically less than 1.0% by weight, preferably less than 0.3% by weight, based on the total dispersion.

The pH of the dispersions essential to the invention is typically less than 9.0, preferably less than 8.0.

The solids content of the polyurethane dispersion is typically 40 to 63% by weight.

In the preparation of the aromatized chewing foams according to the invention, according to process step i), besides the dispersions (I) and foam auxiliaries (III), if appropriate also aromatizing compositions (II), thickeners (IV), and cosmetic additives (V) are co-used.

Aromatizing compositions (II) for the purposes of the present invention comprise sensorily effective substances, which may be volatile (aroma substances) or nonvolatile (taste substances). These compositions (II) are incorporated into the chewing foams according to the invention in amounts such that a sensory effect occurs when the foams are chewed.

The (volatile) aroma substances can be perceived by people both orthonasally and retronasally. The taste substances interact with the taste receptors of the tongue and are responsible for the gustatory (taste) impressions sweet, sour, bitter, salty and umami; in addition, other frequently trigeminal stimuli are also perceived, such as, for example, pungent, burning, cooling, electrifying (“tingling”) or tickling effects.

Usually, the aromatizing compositions (II) comprise at least one aroma substance, preferably 2, 3, 4, 5, 6, 7, 8, 9, 10 or more.

Taste substances for the purposes of the present invention thus include, inter alia, (mucosa-) cooling agents, (mucosa-)warming agents, pungent-tasting substances, sweeteners, sugar substitutes, organic or inorganic acidifiers, such as malic acid, acetic acid, citric acid, tartaric acid and/or phosphoric acid, bitter substances, such as quinine, caffeine, limonene, amarogentin, humolones, lupolones, catechins and/or tannins, and also edible mineral salts, such as sodium chloride, potassium chloride, magnesium chloride and/or sodium phosphates.

Advantageous aroma substances which are suitable as constituent of the aromatized chewing foams are given, for example, in S. Arctander, Perfume and Flavor Chemicals, Vol. I and II, Montclair, N.J. 1969, in-house publisher, or K. Bauer, D. Garbe and H. Surburg, Common Fragrance and Flavor Materials, 4th edition, Wiley-VCH, Weinheim 2001.

Those which may be mentioned by way of example are aliphatic saturated or unsaturated esters, such as ethyl butyrate or allyl capronate; aromatic esters, such as benzyl acetate or methyl salicylate; cyclic alcohols, such as menthol; aliphatic alcohols, such as isoamyl alcohol or 3-octanol; aromatic alcohols, such as benzyl alcohol; aliphatic saturated or unsaturated aldehydes, such as acetaldehyde or isobutyraldehyde; aromatic aldehydes, such as benzaldehyde or vanillin; ketones, such as menthone, carvone; cyclic ethers, such as 4-hydroxy-5-methylfuranone; aromatic ethers, such as p-methoxybenzaldehyde or guaiacol; lactones, such as gamma-decalactone; terpenes, such as limonene, linalool, terpinene, terpineol or citral.

Preferred aroma substances are selected from the group consisting of menthol (preferably l-menthol and/or racemic menthol), anethole, anisole, anisaldehyde, anisyl alcohol, (racemic) neomenthol, eucalyptol (1,8-cineol), menthone (preferably L-menthone), isomenthone (preferably D-isomenthone), isopulegol, menthyl acetate (preferably L-menthyl acetate), menthyl propionate, carvone (preferably (−)-carvone, if appropriate as a constituent of a spearmint oil), methyl salicylate (if appropriate as component of a wintergreen oil), eugenol acetate, isoeugenol methyl ether, beta-homocyclocitral, eugenol, isobutyraldehyde, 3-octanol, dimethyl sulphide, trans-2-hexenal, cis-3-hexenol, 4-terpineol, piperitone, linalool, 8-ocimenyl acetate, isoamyl alcohol, isovaleraldehyde, alpha-pinene, beta-pinene, limonene (preferably D-limonene, if appropriate as constituent of an essential oil), piperitone, trans-sabinene hydrate, methofuran, caryophyllene, germacrene D, cinnamaldehyde, mintlactone, thymol, gamma-octalactone, gamma-nonalactone, gamma-decalactone, (1,3E,5Z)-undecatriene, 2-butanone, ethyl formate, 3-octyl acetate, isoamyl isovalerate, cis- and trans-carvyl acetate, p-cymene, damascenone, damascone, cis-rose oxide, trans-rose oxide, fenchol, acetaldehyde diethyl acetal, 1-ethoxyethyl acetate, cis-4-heptenal, cis-jasmone, methyl dihydrojasmonate, menthyl methyl ether, myrtenyl acetate, 2-phenylethyl alcohol, 2-phenylethyl isobutyrate, 2-phenylethyl isovalerate, geraniol and nerol.

Likewise preferred aroma substances are essential oils and extracts, tinctures and balsams, such as anise oil, basil oil, bergamot oil, bitter almond oil, camphor oil, citronella oil, lemon oil; eucalyptus citriodora oil, eucalyptus oil, fennel oil, grapefruit oil, ginger oil, camomile oil, spearmint oil, cumin oil, limetta oil, mandarin oil, nutmeg oil (in particular nutmeg blossom oil=mace oil), myrrh oil, clove oil, clove blossom oil, orange oil, oregano oil, parsley (seed) oil, peppermint oil, rosemary oil, sage oil (clary sage, Dalmatian or Spanish sage oil), star anise oil, thyme oil, vanilla extract, juniper oil (in particular juniper berry oil), wintergreen oil, cinnamon leaf oil, cinnamon bark oil, and also fractions thereof, and ingredients isolated therefrom.

In order to achieve a refreshing effect in the oral, throat and/or nasal cavity, preference is given to aroma substances from the group consisting of l-menthol, racemic menthol, anethole, anisaldehyde, anisyl alcohol, neomenthol, eucalyptol (1,8-cineol), L-menthone, D-isomenthone, isopulegol, L-menthyl acetate, (−)-carvonc, methyl salicylate, trans-2-hexenal, cis-3-hexenol, 4-terpineol, linalool, 8-ocimenyl acetate, alpha-pinene, D-limonene, (+)-menthofuran, cinnamaldehyde and menthyl methyl ether.

Particularly preferred substances with a refreshing effect in the oral, throat and/or nasal cavity are menthol, menthone, isomenthone, 1,8-cineol (eucalyptol), (−)-carvone, 4-terpineol, thymol, methyl salicylate and L-menthyl methyl ether.

Menthol can be used here in pure form (natural or synthetic) and/or as a constituent of natural oils and/or menthol-containing fractions of natural oils, especially in the form of essential (i.e. obtained by means of steam distillation) oils of certain Mentha species, in particular from Mentha arvensis (corn mint) and from Mentha piperita (peppermint), these include Mentha piperita oils having regional designations of origin of specific areas of cultivation such as Willamette, Yakima and Madras, and also oils of the type of the abovementioned designations. These peppermint oils can be used in natural or else also nature-identical (synthetic) form.

(−)-Carvone can be used here in pure form (natural or synthetic) and/or as a constituent of natural oils and/or menthol-containing fractions of natural oils, especially in the form of essential (i.e. obtained by means of steam distillation) oils of certain Mentha species, in particular from Mentha cardiaca or Mentha spicata.

Anethole can be used here as cis- or trans-anethole or in the form of mixtures of the isomers. Anethole can be used here in pure form (natural or synthetic) and/or as a constituent of natural oils and/or anethole-containing fractions of natural oils, in particular in the form of anise oil, star anise oil or fennel oil or anethole-containing fractions thereof.

Eucalyptol can be used in pure form (natural or synthetic) and/or as a constituent of natural oils and/or eucalyptol-containing fractions of natural oils, for example in the form of bay (leaf) oil, but preferably eucalyptus oils from Eucalyptus fruticetorum and/or Eucalyptus globulus and/or eucalyptol-containing fractions thereof.

Under some circumstances, instead of, or in addition to, the refreshing effect, a cooling effect may also be desired.

Preferred cooling active ingredients used for this purpose are menthone glycerol acetal (trade name: Frescolat® MGA, Symrise GmbH & Co KG, Holzminden, Germany), menthyl lactate (trade name: Frescolat®ML Symrise GmbH & Co KG, Holzminden, Germany; preferably menthyl lactate is 1-menthyl lactate, in particular 1-menthyl l-lactate), substituted menthyl-3-carboxamides (e.g. menthyl-3-carboxylic acid N-ethylamide, also known as WS-3), 2-isopropyl-N-2,3-trimethylbutanamide (also known as WS-23), substituted cyclohexanecarboxamides, 3-menthoxypropane-1,2-diol, 2-hydroxyethylmenthyl carbonate, 2-hydroxypropylmenthyl carbonate, N-acetylglycine menthyl ester, isopulegol, menthyl hydroxycarboxylic esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl-3,6-di- and -trioxaalkanoate, 3-menthyl methoxyacetate, icilin.

Particularly preferred cooling active ingredients are: menthone glycerol acetal, menthyl lactate (preferably 1-menthyl lactate, in particular 1-menthyl l-lactate), substituted menthyl-3-carboxamides (e.g. menthyl-3-carboxylic acid N-ethylamide), 2-isopropyl-N-2,3-trimethylbutanamide, 3-menthoxypropane-1,2-diol, 2-hydroxyethylmenthyl carbonate, 2-hydroxypropylmenthyl carbonate, isopulegol and monomenthyl succinate.

Preferably, the compositions (II) used for the aromatizing have a composition such that they comprise at least one refreshing active ingredient and one cooling active ingredient of the abovementioned type. A preferred mixture of aroma substances therefore comprises 1-menthol and at least one of the abovementioned cooling substances.

It is likewise preferred to configure the aromatizing compositions (II) in such a way that, instead of, or in addition to, a cooling and refreshing effect, they also have a herbal, minty, cinnamon-like, clove-like, eucalyptus, wintergreen and/or fruity character.

Minty includes in particular peppermint and spearmint.

The abovementioned aroma substances can be used here in the aromatizing compositions (II) individually or in any desired mixtures with one another.

Particularly preferably, the aromatizing compositions (II) comprise at least 3, very particularly preferably at least 5, of the abovementioned aroma substances.

Optically active aroma substances can be used here in enantiomerically pure form, or as any desired mixtures of the two enantiomers. The same applies to (E)/(Z)-isomers and diastereomers.

As taste substances, sugar substitutes such as mannitol, sorbitol and sorbitol syrup, isomalt (e.g. Palatinit®), maltitol and maltitol syrup, lactitol, xylitol, erythritol, leucrose, arabinol, arabitol, adonitol, alditol, ducitol, iditol, but also fructooligosaccharides (e.g. Raftilose), oligofructose or polydextrose, for example, may be present in the aromatizing compositions of component (II).

Typical sweeteners such as saccharin (if appropriate as Na, K or Ca salt), aspartame (e.g. NutraSweet®), cyclamate (if appropriate as Na or Ca salt), acesulfame K (e.g. Sunett®), thaumatin, neohesperidin dihydrochalcone, stevioside, rebaudioside A, glycyrrhizin, ultrasweet, osladin, brazzein, miraculin, pentadin, phyllodulcin, dihydrochalcones, arylureas, trisubstituted guanidines, glycyrrhizin, superaspartame, suosan, sucralose (trichlorogalactosucrose, TGS), Alitame, monellin or Neotame® (Sweetners Holdings Inc. USA) may likewise be present, where sucralose has proven particularly advantageous in combination with other sweeteners, in particular with saccharines.

Substances which have a pungent taste and/or stimulate the flow of saliva in the mouth and/or cause a feeling of heat and/or a tingling feeling on the skin or on the mucosae may likewise be present. Examples of such compounds are capsaicin, dihydrocapsaicin, gingcrols, paradols, shogaols, piperin, carboxylic acid N-vanillylamides, in particular nonanoic acid N-vanillylamide, pcllitorin or spilanthol, 2-nonenoic acid amides, in particular 2-noncnoic acid N-isobutylamidc, 2-nonenoic acid N-4-hydroxy-3-methoxyphenylamide, alkyl ethers of 4-hydroxy-3-methoxybenzyl alcohol, in particular 4-hydroxy-3-methoxybenzyl n-butyl ether, alkyl ethers of 4-acyloxy-3-methoxybenzyl alcohol, in particular 4-acetyloxy-3-methoxybenzyl n-butyl ether and 4-acetyloxy-3-methoxybenzyl n-hexyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, (4-hydroxy-3-methoxyphenyl)acetamides, in particular (4-hydroxy-3-methoxyphenyl)acetic acid N-n-octylamide, vanillomandelic acid alkylamides, ferulic acid phenethylamides, nicotin-aldehyde, methyl nicotinate, propyl nicotinate, 2-butoxyethyl nicotinate, benzyl nicotinate, 1-acetoxychavicol, polygodial and isodrimeninol, further preferably cis- and/or trans-pellitorin according to WO 2004/000787 and WO 2004/043906, alkenecarboxylic acid N-alkylamides according to WO 2005/044778, mandelic acid alkylamides according to WO 03/106404 or alkyloxyalkanoic acid amides according to WO 2006/003210.

Preferred natural extracts that have a pungent taste and/or cause a feeling of heat and/or a tingling feeling on the skin or on the mucosae are those from paprika, pepper (e.g. capsicum extract), chilli pepper, ginger root, Aframomum melgueta, Spilanthes acmella, Kaempferia galanga or Alpinia galanga.

In addition, substances for masking one or more unpleasant taste impressions, in particular a bitter, astringent and/or metallic taste impression or aftertaste, may be present. Examples which may be mentioned are lactisol [2O-(4-methoxyphenyl)lactic acid] (cf. U.S. Pat. No. 5,045,336), 2,4-dihydroxybenzoic acid potassium salt (cf. U.S. Pat. No. 5,643,941), ginger extracts (cf. GB 2,380,936), neohesperidin dihydrochalcone (cf. Manufacturing Chemist 2000, July issue, pp. 16-17), flavones (2-phenylchrom-2-en-4-ones) (cf. U.S. Pat. No. 5,580,545), certain nucleotides, such as cytidine 5′-monophosphate (CMP) (cf. US 2002/0177576), sodium salts, such as sodium chloride, sodium citrate, sodium acetate and sodium lactate (cf. Nature, 1997, volume 387, p. 563), lipoproteins from β-lactoglobulin and phosphatidic acid (cf. EP-A 635 218), neodiosmin [5,7-dihydroxy-2-(4-methoxy-3-hydroxyphenyl)-7-0-neohesperidosylchrom-2-en-4-one] (cf. U.S. Pat. No. 4,154,862), preferably hydroxyflavanones according to EP 1 258 200, in turn here preferably 2-(4-hydroxyphenyl)-5,7-dihydroxychroman-4-one (naringenin), 2-(3,4-dihydroxyphenyl)-5,7-dihydroxychroman-4-one (eriodictyol), 2-(3,4-dihydroxyphenyl)-5-hydroxy-7-methoxychroman-4-one (eriodictyol 7-methyl ether), 2-(3,4-dihydroxyphenyl)-7-hydroxy-5-methoxychroman-4-one (eriodictyol 5-methyl ether) and 2-(4-hydroxy-3-methoxyphenyl)-5,7-dihydroxychroman-4-one (homoeriodictyol), their (2S)- or (2R)-enantiomers or mixtures of the same, and also their monovalent or polyvalent phenolate salts with Na⁺, K⁺, NH₄⁺, Ca²⁺, Mg²⁺ or Al³⁺ as counter cations, or γ-aminobutyric acid (4-aminobutanoic acid, as neutral form (“internal salt”) or in the carboxylate or ammonium form) according to WO 2005/096841.

It has also been established that aromatized chewing foams according to the invention comprising a combination of (a) one or more physiological cooling active ingredients, in particular of Frescolat® ML (menthyl lactate), menthyl ethylene glycol carbonate and/or menthyl propylene glycol carbonate preferably in the form of Optacool® (Symrisc GmbH&CO KG, Holzminden, Germany), comprising a combination of menthyl ethylene glycol carbonate and menthyl propylene glycol carbonate) and (b) trans-pellitorin ((2E,4)-decadienoic acid N-isobutylamide), a saliva-stimulating and slightly tingling aroma substance) a significant taste improvement of the aromatized chewing foams is achieved, said combinations preferably being used in process step i); similar positive effects are observed when using said combination in process step vi) and in particular as a constituent of the aroma coating (VII). The use of these taste substances in combination with saccharin and sucralose then produce a particularly pleasant, fresh feel in the mouth.

The abovementioned aroma substances and taste substances of the aromatizing compositions (II) are, preferably before being incorporated into the chewing foams, firstly incorporated into a matrix (carrier substance) suitable for foods and items consumed for pleasure, e.g. in the form of emulsions, liposomes, e.g. starting from phosphatidylcholine, microspheres, nanospheres, or else in capsules, granules or extrudates. Preferably, the matrix here is chosen in each case such that the taste substances and/or aroma substances are released from the matrix in a delayed manner, so that a long-lasting effect is achieved.

Preferred matrices are selected here from the following group: polysaccharides such as starch, starch derivatives, cellulose or cellulose derivatives (such as hydroxypropylcellulose), alginates, gellan gum, agar or carrageen, natural fats, natural waxes such as beeswax, carnauba wax, proteins such as gelatine, complexing agents such as cyclodextrins or cyclodextrin derivatives, preferably beta-cyclodextrin.

The loading of the matrices with taste substances and/or aroma substances to be used according to the invention can vary according to requirement and the desired sensory profile. Usually, the loading of taste substances and/or aroma substances is 1 to 60% by weight, preferably 5 to 40% by weight, based on the total weight of matrix (carrier substance) and taste substances and/or aroma substances.

The stated amounts of the aromatizing compositions (II) always refer here to the total mass of the taste substances and/or aroma substances used. These data include any amounts of matrices or carrier materials for the taste substances and/or aroma substances present.

In addition, it has proven advantageous to convert the taste substances and/or aroma substances of the aromatizing component (II) into a spray-dried form before incorporating them into the chewing foams. Preferred matrices used here are starches, degraded starches, chemically or physically modified starches, modified celluloses, gum arabic, ghatti gum, tragacanth, karaya, carrageenan, guar seed flour, carob seed flour, alginates (e.g. Na alginate), pectin, inulin, xanthan gum or maltodextrins individually or in any desired mixtures with one another. Preference is given to using film-forming substances which are to be classified as noncariogenic and therefore as tooth-friendly. Particularly preferred carrier substances for the provision of spray-dried taste substances and/or aroma substances are maltodextrins, and mixtures of maltodextrins and gum arabic, where in each case maltodextrins with DE values in the range 15 to 20 are in turn advantageous.

The degree of decomposition of the starch is measured by the characteristic “dextrose equivalent” (DE), which can assume the limiting value 0 for the long-chain glucose polymer and 100 for pure glucose.

The encapsulation of taste substances and/or aroma substances by means of spray-drying is known to the person skilled in the art, and described, for example, in U.S. Pat. No. 3,159,585, U.S. Pat. No. 3,971,852, U.S. Pat. No. 4,532,145 or U.S. Pat. No. 5,124,162. Spray-dried aromas are commercially available in many different flavour directions and particle sizes.

An exemplary aromatizing composition (II) can have the following quantitative ratios of various individual substances (taste substances and aroma substances), where the sum of the individual components preferably add up to 100% by weight:

| | |
|---|---|
| 1 to 68% by weight | menthols and menthol derivatives including the |
| | racemic form |
| 5 to 30% by weight | natural or synthetic peppermint oils of various |
| | provinces |
| 5 to 30% by weight | natural or synthetic arvensis oils (corn mint oils |
| | in USA) |
| 2 to 20% by weight | cooling substances such as Optacool ®, WS 3, |
| | WS 23, |
| 2 to 15% by weight | so-called “sensates”, which have a slightly |
| | tingling and flavour-enhancing effect and may |
| | stimulate saliva (e.g. Optaflow - transpelletorin) |
| 5 to 20% by weight | anethols, or fennel oil, anise oil, |
| 5 to 20% by weight | citrus oils, herbaceous oils, |
| 2 to 15% by weight | methyl salicylate |
| 2 to 20% by weight | cinnamaldehyde, cinnamon bark oil, |
| 2 to 20% by weight | eucalyptus oil |
| 2 to 20% by weight | clove oil and/or eugenol. |

Foam auxiliaries (III) which may be used are all foaming agents and/or foam stabilizers known to the person skilled in the art. Suitable foam auxiliaries (III) are standard commercial materials, such as, for example, water-soluble fatty acid amides, sulphosuccinamides, hydrocarbon sulphonates, hydrocarbon sulphates, fatty acid salts, where the lipophilic radical preferably contains 12 to 24 carbon atoms, alkyl polyglycosides etc.

Alkyl polyglycosides per se are obtainable by the methods know to the person skilled in the art, e.g. by reacting relatively long-chain monoalcohols with mono-, di- or polysaccharides (Kirk-Othmer Encyclopedia of Chemical Technology, John Wiley & Sons, vol. 24, p. 29). The relatively long-chain monoalcohols, which may optionally also be branched, preferably have 4 to 22 carbon atoms, preferably 8 to 18 carbon atoms and particularly preferably 10 to 12 carbon atoms in an alkyl radical. Specifically, as relatively long-chain monoalcohols, mention may be made of 1-butanol, 1-propanol, 1-hexanol, 1-octanol, 2-ethylhexanol, 1-decanol, 1-undecanol, 1-dodecanol (lauryl alcohol), 1-tetradecanol (myristyl alcohol) and 1-octadecanol (stearyl alcohol). It is of course also possible to use mixtures of the specified relatively long-chain monoalcohols.

Preferably, these alkyl polyglycosides have structures derived from glucose.

Particular preference is given to using alkyl polyglycosides of the formula (I):

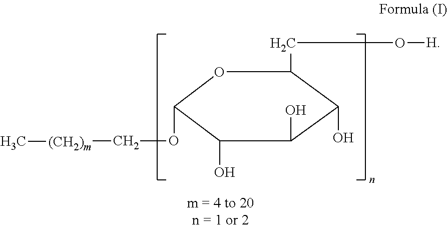

Preferably, m is a number from 6 to 20, particularly preferably 10 to 16.

The alkyl polyglycosides preferably have an HLB value of less than 20, particularly preferably of less than 16 and very particularly preferably of less than 14, the HLB being calculated by the formula HLB=20·Mh/M, where Mh is the molar mass of the hydrophilic fraction of a molecule and M is the molar mass of the total molecule (Griffin, W. C.: Classification of surface active agents by HLB, J. Soc. Cosmet. Chem. 1, 1949).

Preferred foam auxiliaries (III) are alkanesulphonates or alkane sulphates having 12 to 22 carbon atoms in the hydrocarbon radical, alkyl polygylcosides of the formula (I) and fatty acid salts, and mixtures thereof.

Particularly preferred foam stabilizers (III) are sodium lauryl sulphate, alkyl polyglycosides, sulphosuccinamides and/or ammonium stearate, and mixtures thereof.

Thickeners (IV) for the purposes of the invention are compounds which allow the viscosity of the resulting mixture of I-V to be adjusted such that the generation and processing of the polymer foam is favoured. Suitable thickeners are standard commercial thickeners such as, for example, natural organic thickeners, e.g. dextrins or starch, organically modified natural substances, e.g. cellulose ethers or hydroxyethylcellulose, organically fully synthetic substances, e.g. polyacrylic acids, polyvinylpyrrolidones, poly(meth)acrylic compounds or polyurethanes (associative thickeners), and inorganic thickeners, e.g. bentonites or silicas. Preference is given to using organically fully synthetic thickeners. Particular preference is given to using acrylate thickeners which, if appropriate, are further diluted with water before being added.

Examples of standard commercial thickeners are Mirox® AM (BGB Stockhausen GmbH, Krefeld, Germany), Walocel® MT 6000 PV (Wolff Cellulosics GmbH & Co KG, Walsrode, Germany), Rheolate® 255 (Elementies Specialities, Gent, Belgium), Collacral®VL (BASF AG, Ludwigshafen, Germany), Aristoflex® AVL (Clariant, Sulzbach, Germany), etc.

In a preferred embodiment of the invention, the use of a thickener (IV) is dispensed with.

Cosmetic additives (V) for the purposes of the invention are, for example, preservatives, abrasives (polishing agents), antibacterial agents, anti-inflammatory agents, irritation-preventing agents, irritation-suppressing agents, antimicrobial agents, antioxidants, astringents, antistatics, binders, (mineral) fillers, buffers, carrier materials, chelators (chelating agents), cleaning agents, care agents, surface-active substances, emulsifiers, enzymes, fibres, film formers, fixatives, foam formers, substances for preventing foaming, foam boosters, gelling agents, gel-forming agents, moisturizers, moistening substances, humectant substances, bleaching agents, lightening agents (e.g. hydrogen peroxide), impregnating agents, friction-reducing agents, lubricants, opacifiers, plasticizing agents, covering agents, shine agents, silicones, mucosae-calming agents, mucosae-cleaning agents, mucosae-care agents, mucosac-healing agents, mucosac-protecting agents, stabilizers, suspension agents, vitamins, fatty oils, waxes, fats, phospholipids, saturated fatty acids, mono- or polyunsaturated fatty acids, polyhydroxy fatty acids, liquefiers, dyes, colour-protecting agents, pigments, surfactants, silicone derivatives, polyols, organic solvents, silicas, calcium carbonate, calcium hydrogenphosphate, aluminium oxide, fluorides, salts of zinc, tin, potassium, sodium and strontium, pyrophosphates, hydroxyapatites.

Antioxidants or substances with an antioxidative effect of component (V) are tocopherols and derivatives thereof, tocotrienols, flavonoids, ascorbic acid and its salts, alpha-hydroxy acids (e.g. citric acid, lactic acid, malic acid, tartaric acid) and the Na, K and Ca salts thereof, ingredients, extracts and fractions thereof isolated from plants, e.g. from tea, green tea, algae, grape seeds, wheat germ, rosemary, oregano; flavonoids, quercetin, phenolic benzylamines. Furthermore, suitable antioxidants are propyl gallate, octyl gallate, dodecyl gallate, butylhydroxyanisol (BHA, E320), butylhydroxytoluene (BHT, 2,6-di-tert-butyl-4-methylphenol, E321), lecithins, mono- and diglycerides of edible fatty acids esterified with citric acid, orthophosphates and Na, K and Ca salts of monophosphoric acid, and ascorbyl palmitate.

Dyes or pigments of component (V) which may be present are: lactoflavin (riboflavin), beta-carotene, riboflavin-5′-phosphate, alpha-carotene, gamma-carotene, cantaxanthin, erythrosin, curcumin, quinoline yellow, yellow orange S, tartrazine, bixin, norbixin (Annatto, Orlean), capsanthin, capsorubin, lycopene, beta-apo-8′-carotenal, beta-apo-8′-carotenic acid ethyl ester, xantophylls (flavoxanthin, lutein, kryptoxanthin, rubixanthin, violaxanthin, rodoxanthin), true carmine (carminic acid, cochineal), azorubin, cochineal red A (Ponceau 4 R), beetroot red, betanene, anthocyans, amaranth, patent blue V, indigotin 1 (indigo carmine), chlorophylls, copper compounds of chlorophylls, brilliant acid green BS (lissamine green), brilliant black BN, Carbo medicinalis vegetabilis, titanium dioxide, iron oxides and hydroxides, calcium carbonate, aluminium, silver, gold, ruby pigment BK (lithol ruby BK), methyl violet B, victoria blue R, victoria blue B, acilan brilliant blue FFR (brilliant wool blue FFR), naphthol green B, acilan true green 10 G (alkali true green 10 G), Ceres yellow GRN, Sudan blue II, ultramarine, phthalocyanine blue, phthalocyanine green, true acid violet R. It is also possible to use further naturally produced extracts (e.g. paprika extract, black carrot extract, red cabbage extract) for colouring purposes. Good results have also been achieved with the colours cited below, the so-called aluminium lakes: FD & C Yellow 5 Lake, FD & C Blue 2 Lake, FD & C Blue 1 Lake, Tartrazine Lake, Quinoline Yellow Lake, FD & C Yellow 6 Lake, FD & C Red 40 Lake, Sunset Yellow Lake, Carmoisine Lake, Amaranth Lake, Ponceau 4R Lake, Erythrosyne Lake, Red 2G Lake, Allura Red Lake, Patent Blue V Lake, Indigo Carmine Lake, Brilliant Blue Lake, Brown HT Lake, Black PN Lake, Green S Lake and mixtures thereof, dyes such as E133 brilliant blue FCF alone or in combination with titanium dioxide. The dyes are also known under the following numbers: E 122, E 120, E 123, E 124, E 127, E 128, E 129 E 131, E 132, E 133, E 140, E 141, E 142, E 150, E 151, E 153.

Suitable (mineral) fillers of component (V) are, for example, calcium carbonate, titanium dioxide, silicon dioxide, talc, aluminium oxide, dicalcium phosphate, tricalcium phosphate, magnesium hydroxide and mixtures thereof.

Antimicrobial active ingredients for improving oral hygiene may be hydrophilic, amphoteric or hydrophobic in nature. Examples of such antimicrobial active ingredients are: triclosan, chlorhexidine and salts thereof (e.g. acetate, gluconate or hydrochloride thereof), peroxides, phenols and salts thereof, domiphen bromide (phenododecinium bromide), bromochlorophen, Zn salts, chlorophylls, Cu salts, Cu gluconate, Cu chlorophyll, sodium lauryl sulphate, quaternary monoammonium salts, such as cocoalkylbenzyldimethylammonium chloride or else pyridinium salts, such as cetylpyridinium chloride. Besides individual active ingredients, it is also possible to use mixtures of active ingredients or natural extracts or fractions thereof comprising active ingredients, such as, for example, those obtainable from neem, berberis, fennel, green tea, marigold, camomile, rosemary, thyme, propolis or turmeric.

Preferred cosmetic additives (V) are emulsifiers (e.g. lecithins, diacylglycerols, gum arabic), stabilizers (e.g. carageenan, alginate), preservatives (e.g. benzoic acid, sorbic acid), antioxidants (e.g. tocopherol, ascorbic acid), chelators (e.g. citric acid), plant extracts, natural or synthetic dyes or colour pigments (e.g. carotenoids, flavonoids, anthocyans, chlorophyll and derivatives thereof) and/or antimicrobial active ingredients.

In a preferred embodiment, the components (II) and (V) of the chewing foams are arranged such that they are suitable for oral hygiene products or dental care compositions. In order to achieve this aim, the following are preferably used: abrasives (or polishing agents), such as silicas, calcium carbonates, calcium phosphates, aluminum oxides and/or hydroxylapatites; surface-active substances, such as sodium lauryl sulphate, sodium lauryl sarcosinate and/or cocamidopropylbetaine; humectants, such as glycerol and/or sorbitol; sweeteners, such as saccharin taste correctors for unpleasant taste impressions, taste correctors for generally not unpleasant taste impressions, taste-modulating substances, such as inositol phosphate, nucleotides, such as guanosine monophosphate, adenosine monophosphate or other substances, such as sodium glutamate or 2-phenoxypropionic acid; carboxymethylcellulose; polyethylene glycols; carrageenan and/or Laponite®; active ingredients, such as sodium fluoride, sodium monofluorophosphate, tin difluoride, quaternary ammonium fluorides, zinc citrate, zinc sulphate, tin pyrophosphate, tin dichloride, mixtures of various pyrophosphates, triclosan, cetylpyridinium chloride, aluminium lactate, potassium citrate, potassium nitrate, potassium chloride, strontium chloride, hydrogen peroxide and/or sodium bicarbonate.

In addition, for this purpose, it is possible to use substances for controlling or preventing plaque, tartar or caries, and also those for controlling or preventing mouth odour, as described in U.S. Pat. No. 5,043,154. By way of example, mention may be made of Zn salts, such as Zn citrate, Zn fluoride, Sn salts, such as Sn fluorides, Cu salts, fluorides, e.g. amine fluorides, alkali metal fluorides, such as Na fluoride, alkaline earth metal fluorides, ammonium fluoride, phosphates, pyrophosphates, fluorophosphates, such as Na mono fluorophosphate, Al monofluorophosphate and Al difluorophosphate, alpha-ionone, geraniol, thymol, isomenthyl acetate, panthenol (provitamin B5), xylitol, allantoin, niacinamide (vitamin B3), tocopheryl acetate (vitamin E actetate), poloxamer.

Preferably, the aromatized chewing foams according to the invention are configured such that they are free from cariogenic substances, such as sucrose, glucose, lactose, hydrolysed lactose, sorbose, arabinose, xylose, mannose, maltose, galactose, maltotriose and fructose; or such that they do not exceed the critical value of pH 5.7 during use as intended when carrying out an in vivo plaque pH test (Imfeld, T.; Monographs in Oral Science, vol. 11, 1983 Basel: Karper).

In the method according to the invention, 80 to 99.5% by weight of the polyurethane dispersion (I), 0.1 to 30% by weight of component (II), 0 to 10% by weight of component (III), 0 to 10% by weight of component (IV) and 0 to 15% by weight of component (V) are used, where the quantitative data are based on the corresponding anhydrous components (I) to (V) and the sum of the anhydrous individual components preferably adds up to 100% by weight.

Preference is given to 80 to 99.5% by weight of the polyurethane dispersion (I), 0.5 to 15% by weight of component (II), 0.1 to 10% by weight of component (III), 0 to 3% by weight of component (IV) and 0 to 10% by weight of component (V), where the quantitative data are based on the corresponding anhydrous components (I) to (V) and the sum of the anhydrous individual components preferably adds up to 100% by weight.

In the process according to the invention, according to process step ii), the foaming can take place by introducing air and/or under the action of corresponding shear energy (e.g. mechanical stirring) or by means of standard commercial blowing agents. Preference is given to introducing air under the action of corresponding shear energy, e.g. through use of standard commercial foam aggregates (e.g. Hansa mixer, Hansa Industrie-Mixer GmbH&Co. KG, Stuhr, Germany or Top Mix Krups 3 mix 8008, Krups GmbH, Offenbach, Germany).

Thus, the aromatizing compositions can, for example, also already be present in the polyurethane-polyurea dispersion (I). Division of the aromatizing composition (II) and the addition at various points in the preparation process is also possible.

Accordingly, it is likewise possible to additionally or alternatively treat the dried chewing foams with aromatizing compositions (II).

In a preferred embodiment of the preparation process according to the invention, an aromatizing composition (II) is added in step i) before the foaming in step ii) and a further aromatizing composition (II) is applied in the form of an aqueous suspension (slurry) to the surface of the ready-shaped and dried chewing foams in process step vi).

According to process step iii) according to the invention, the foamed composition can be applied in highly diverse ways to various surfaces or in moulds, such as, for example, by pouring, knife-coating, rolling, coating, injection-moulding, spraying or extrusion. Preference is given to pouring and knife-coating. Particular preference is given to pouring, where flat mats with a thickness of from 3 mm to 25 mm, preferably 5 mm to 20 mm, particularly preferably 8 mm to 18 mm are prepared. In order to ensure a large drying surface, water-permeable or steam-permeable substrates or moulding materials are preferably used (e.g. release paper: VEZ mat, Sappi, Brussels, Belgium; water-permeable plastic fabric, e.g. Sefar Tetex Mono 08-1050-K039, or Sefar Propyltex 05-1000/45 1 mm mesh width, Sefar GmbH, Wasserburg, Germany, etc.).

The aromatized chewing foams can also be applied in a plurality of layers, for example for producing particularly tall foam pads, to a very wide variety of substrates, or be poured into moulds. Preferably, a multilayer structure is dispensed with.

Whereas the foamed compositions before drying have a foam density of from 200 to 900 g/l, preferably 250 to 600 g/l, the density of the resulting chewing foams after drying is preferably 50 to 700 g/l, particularly preferably 200 to 550 g/l.

For the purposes of the present invention, drying means reducing the water content in a foam to be dried and/or in the applied aromatizing coating.

In connection with the present inventions, moist means a water fraction in the foam material of at least 10% by weight, preferably 15 to 60% by weight, particularly preferably 35 to 60% by weight, based on the mass of the foam material.

In the case of the coating (VI), moist means a water fraction of at least 10% by weight, preferably 15 to 60% by weight, particularly preferably 35 to 60% by weight, based on the mass of the coating material (VI).

In the case of the aromatizing coating (VII), moist means a water fraction of from 15 to 99% by weight, based on the aromatizing coating (VII).

The actual drying of the foam mass preferably takes place through the action of microwave radiation. For the purposes of the invention, microwave radiation is understood as meaning electromagnetic radiation in the wavelength range from 300 MHz to 300 GHz. Preference is given to radiations in the frequency ranges 2.0 to 3.0 GHz and 0.8 to 1.5 GHz. Particularly preferred frequencies are 2.2 to 2.6 and 0.85 to 1.0 GHz. Very particular preference is given to the frequencies 2.45 GHz (±0.1 GHz) and 0.915 GHz (±0.05 GHz).

The power introduced at the abovementioned frequencies is preferably 250 to 6000 W, particularly preferably 500 to 4000 W per kilogram of the foam to be dried.

For the microwave-based drying of the moist foam, the shaped foams are dried, if necessary at elevated temperature between 20° C. and 100° C., over the course of from 1 to 200 minutes, preferably from 2 to 60 minutes, most preferably over the course of from 15 to 45 minutes.

In addition, it is possible, besides the exclusive use of microwave radiation, to also use a combination of microwave radiation and conventional drying, such as IR radiation and/or convection drying. Here, it is unimportant whether the two drying methods are used in parallel or in succession.

Following the process according to the invention, in step v), from the foam mats obtained as described above, the chewing foams are converted to the desired arbitrary shape with maximum dimensions of 25×25×25 mm (width×height×length), preferably 20×20×20 mm (width×height×length) preferably by means of a cutting process and/or punching process. Cutting/punching processes which may be used are all processes known to the person skilled in the art, such as, for example: hot-wire cutting, laser cutting, water jet cutting, roll punching, etc. Particular preference is given to using a punching process.

According to process step vi), the chewing foam blanks obtained after the cutting/punching process can additionally also be passed to a surface refining.

As surface refining, a further coating (VI) can be applied to improve the optical properties and the bite resistance.

In a particular embodiment, the coating (VI) consists of a polyurethane dispersion (I) which also serves as a basis for producing the chewing foams.

The coating (VI) can be applied by customary coating technologies, such as, for example, dipping, spraying, coating, drum coating, fluidized-bed coating etc.

This coating (VI) can additionally comprise, if appropriate, aromatizing compositions (II) and dyes and/or pigments and/or further cosmetic additives (V).

It is also possible to sprinkle or to sieve such aromatizing compositions (II) and/or cosmetic additives (V) in suitable application forms (e.g. as powder, granules, extrudate, capsules, etc.) on the still-moist polyurethane coating (VI).

For the convection drying of the coating (VI), the shaped chewing foams are preferably dried at elevated temperature between 60° C. and 140° C., preferably 80° C. to 135° C., over the course of from 2 to 60 minutes, preferably 10 to 40 minutes.

In addition, it is possible, in the course of the surface refining, besides the exclusive use of convection drying processes, to also use a combination of convection drying and microwave radiation or other drying processes, such as IR radiation and/or microwave drying. Here, it is unimportant whether the different types of drying are used in parallel or in succession.

If in the course of surface refining, a coating (VI) is applied, then it is likewise possible to also apply a further coating (VII) after drying the coating (VI).

In so doing, it is possible, through a first coating (VI), to increase the mechanical properties, such as the bite resistance, and, in the course of the second coating (VII), to achieve optimum aromatization.

For this, this second coating (VII) is provided with an aromatizing composition (II). This aroma coating (VII) is applied in a concentration of from 1 to 50% by weight, preferably 5-35% by weight, most preferably 5 to 25% by weight, based on the weight of the chewing foam.

This aroma coating (VII) can be applied by customary coating techniques, such as, for example, coating, dipping, spraying, knife-coating, pouring, etc. Depending on the composition of the aroma coating (VII), it may be necessary to dry this during and/or after the application at an elevated temperature of between 20 and 180° C., preferably of between 30 and 140° C., most preferably of between 40 and 140° C., in detail of between 40 to 80° C., for 1 to 60 minutes, preferably 3 to 40 minutes and most preferably 3 to 20 minutes.

The aroma coating (VII) can consist solely of aromatizing compositions (II) and/or of aromatizing compositions (II), cosmetic auxiliaries (V) and a suitable binder, such as, for example, a polyurethane dispersion (I), gelatin, alginates, carrageen, gum arabic, xanthan gum, celluloses and its derivatives, xylitol and also other sugar substitutes. Preferred binders are those which are fit for foods and are thus suitable for consumption. In one particular embodiment, the aroma coating (VII) has the following composition, the individual amounts preferably adding up to 100% by weight:

30 to 89% by weight of water
10 to 30% by weight of sugar substitute, such as, for example, sorbitol, mannitol, xylitol or isomalt
0 to 3% by weight of sweetener, such as, for example, saccharin or Na saccharin
0 to 40% by weight of gum arabic powder
1 to 60% by weight of aromatizing composition (II) in spray-dried application form
0 to 2% by weight of dyes (V).

To prepare this aroma coating (VII), the ingredients are stirred together at room temperature to give a homogeneous aqueous suspension (slurry).

In a further preferred embodiment, the aroma coating (VII) has the following composition, the individual amounts preferably adding up to 100% by weight:

80 to 98.9% by weight of water
0.1 to 3% by weight of carrageen
1 to 5% by weight of aromatizing composition (II) in liquid application form.

To prepare this aroma coating (VII), the carrageen is homogenized in water at elevated temperature (50 to 90° C., preferably 70 to 90° C.) and then the aromatizing composition (II) is added until a homogeneous aqueous suspension (slurry) has formed.

In a further preferred embodiment, the aroma coating (VII) has the following composition, the individual amounts preferably adding up to 100% by weight:

70 to 92.5% by weight of water
5 to 10% by weight of alginate
0.5 to 3% by weight of microcrystalline cellullose
0.5 to 3% by weight of glycerol
0.5 to 3% by weight of sugar substitute, such as, for example, sorbitol, mannitol, xylitol or isomalt
1 to 7% by weight of aromatizing composition (II) in liquid form.

To prepare this aroma coating (VII), the ingredients are stirred together at room temperature to give a homogeneous aqueous suspension (slurry).

After applying the aroma composition (VII) to one or more sides of the shaped chewing foams, the latter are dried by convection as mentioned above.

It is also possible, besides the exclusive use of convection drying processes, to also use a combination of convection drying and microwave radiation or other drying processes, such as IR radiation and/or microwave drying. In this connection, it is unimportant whether the different types of drying are used in parallel or in succession.

The aromatized chewing foams prepared by the process according to the invention have excellent mechanical properties; during a normal chewing operation of up to 3 minutes, they cannot be destroyed, return to their original shape after the chewing operation and thus have the ability to clean the chewing surfaces and teeth sides, have a pleasant taste, refresh the oral cavity region (oral, throat and/or nasal cavity) and do not stick to floor coverings.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

### Substances and Abbreviations Used

Diaminosulphonate: NH₂—CH₂CH₂—NH—CH₂CH₂—SO₃Na (45% strength in water)
Desmophen® C2200: polycarbonate polyol, OH number 56 mg KOH/g, number-average molecular weight 2000 g/mol (Bayer MaterialScience AG, Leverkusen, Germany)

PolyTHF® 2000: polytetramethylene glycol polyol, OH number 56 mg KOH/g, number-average molecular weight 2000 g/mol (BASF AG,

### Ludwigshafen, Germany)

PolyTHF® 1000: polytetramethylene glycol polyol, OH number 112 mg

KOH/g, number-average molecular weight 1000 g/mol

### (BASF AG, Ludwigshafen, Germany)

Polyether LB 25: (monofunctional polyether based on ethylene oxide/propylene oxide, number-average molecular weight 2250 g/mol, OH number 25 mg KOH/g (Bayer MaterialScience AG, Leverkusen, Germany)
Stokal® STA: aqueous ammonium stearate solution (Bozzetto GmbH, Krefeld, Germany)
Loxanol® K12P sodium lauryl sulphate ether (Cognis GmbH, Dusseldorf Germany)
Plantacare® 1200 UP alkyl polyglycosides (Cognis GmbH, Dusseldorf, Germany)
Na saccharin: sweetener (Merck, Darmstadt KGaA Germany)
L-menthol Freeflow (PN 600129): l-menthol free-flowing (mixture consisting of 1-menthol and 1% by weight of silicon dioxide) (Symrise, Holzminden, Germany)
Peppermint aroma (PN 134229): spray-dried peppermint oil with up to 40% by weight loading based on gum arabic (Symrise, Holzminden, Germany)
Optamint® peppermint (PN 225977): liquid aroma (Symrise, Holzminden, Germany)
Evogran® Spearmint (PN 356729): encapsulated mint aroma (Symrise, Holzminden, Germany)
Optacool®: mixture of different physiological cooling active ingredients (Symrise, Holzminden Germany)
Viscarin® GP 109F carrageen (FMC Biopolymers, Philadelphia, USA)
Protanal® GP 2650 alginate (FMC Biopolymers, Philadelphia, USA)
Avicel® FD100 MCC microcrystalline cellulose (FMC Biopolymers, Philadelphia, USA)

### Example 1

### Preparation of a Polyurethane-Polyurea Dispersion (I)

761.3 g of Desmophen® C2200, 987.0 g of PolyTHF® 2000, 375.4 g of PolyTHF® 1000 and 53.2 g of polyether LB 25 were heated to 70° C. Then at 70° C., over the course of 5 min, a mixture of 237.0 g of hexamethylene diisocyanate and 313.2 g of isophorone diisocyanate was added, and stirring was carried out under reflux until the theoretical NCO value had been reached. The finished prepolymer was dissolved with 4850 g of acetone at 50° C. and then a solution of 1.8 g 25.1 g of ethylenediamine, 61.7 g of diaminosulphonate, 116.5 g of isophoronediamine and 1030 g of water was metered in over the course of 10 min. The afterstirring time was 10 min. The mixture was then dispersed by adding 1061 g of water. Removal of the solvent by distillation in vacuo followed, giving a storage-stable dispersion with a solids content of 57%.

### Example 2

### Preparation of a Coating Material (VI)

100 g of polyurethane dispersion from Example 1, 3 g of a 0.2% strength aqueous solution of Na saccharin and 3 g of L-menthol Freeflow PN 600129 are homogeneously together at room temperature.

### Example 3

### Preparation According to the Invention of an Aromatized Chewing Foam

1000 g of the dispersion (I) obtained from Example 1 were mixed with 9 g of Loxanol® K12P (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All of the sides of the cubes were painted with the coating material (VI) prepared in Example 2 using a brush and then dried in a convection oven at 130° C. for 30 minutes.

### Example 4

### Preparation According to the Invention of an Aromatized Chewing Foam

1000 g of the dispersion (I) obtained from Example 1 were mixed with 10 g of Plantacare® 1200 UP (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the coating material (VI) prepared in Example 2 using a brush.

Then, about 0.1 g of peppermint aroma PN 134229 (II) was applied to the still-damp coating (VI) by sieving. The coated cubes were then dried in a convection oven at 130° C. for 30 minutes.

### Example 5

### Preparation According to the Invention of an Aromatized Chewing Foam

1000 g of the dispersion (I) obtained from Example 1 were mixed with 9 g of Loxanol® K12P (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the coating material prepared in Example 2 with the help of a brush and then dried in a convection oven at 130° C. for 25 minutes.

The cubes were then sprayed with 0.1 g of an aroma coating (VII) of composition: 65 g of water, 20 g of sorbitol, 15 g of peppermint aroma PN 134229 and 0.2 g of sodium saccharin from all sides. The cubes were then dried in a convection oven at 130° C. for 5 minutes.

### Example 6

### Preparation According to the Invention of an Aromatized Chewing Foam

1000 g of the dispersion (I) obtained from Example 1 were mixed with 9 g of Loxanol® K12P (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the coating material prepared in Example 2 with the help of a brush and then dried in a convection oven at 130° C. for 25 minutes.

The cubes were then painted with 0.1 g of an aroma coating (VII) of composition: 35 g of water, 20 g of sorbitol, 50 g of peppermint aroma PN 134229 and 0.2 g of sodium saccharin on one side using a brush. The cubes were then dried in a convection oven at 130° C. for 5 minutes.

### Example 7

### Preparation According to the Invention of an Aromatized Chewing Foam

1000 g of the dispersion (I) obtained from Example 1 were mixed with 10 g of Plantacare® 1200 UP (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the coating material prepared in Example 2 using a brush. Then, about 0.1 g of Evogran Spearmint PN 346729 (II) was applied to the still-damp coating (VI) by sieving. The coated cubes were then dried in a convection oven at 130° C. for 30 minutes.

### Example 8

### Preparation According to the Invention of an Aromatized Chewing Foam

1000 g of the dispersion (I) obtained from Example 1 were mixed with 10 g of Plantacare® 1200 UP (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/1. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the binder mixture prepared in Example 2 using a brush and then dried in a convection oven at 130° C. for 25 minutes.

The cubes were then painted with 0.1 g of an aroma coating (VII) of composition: 97 g of water, 1 g Viscarin® GP 109F and 2 g of Optamint® peppermint aroma PN 225977, on all sides using a brush. The aroma coating (VII) was prepared by mixing the ingredients and stirring at 82° C. The cubes were then dried in a convection oven at 130° C. for 5 minutes.

### Example 9

### Preparation According to the Invention of an Aromatized Chewing Foam

1000 g of the dispersion (I) obtained from Example 1 were mixed with 10 g of Plantacare® 1200 UP (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the coating material prepared in Example 2 using a brush and then dried in a convection oven at 130° C. for 25 minutes.

The cubes were then painted with 0.1 g of an aroma coating (VII) of composition: 81.2 g of water, 8.75 Protanal® GP 2650 g, 1.75 g of Avicel® FD100 MC, 1.7 g of sorbitol, 1.6 g of glycerol and 5.0 g of Optamint® peppermint aroma PN 225977 on all sides using a brush. The aroma coating (VII) was prepared by mixing the ingredients and stirring at room temperature. The cubes were then dried in a convection oven at 130° C. for 15 minutes.

All of the aromatized chewing foams prepared according to the invention are bite-resistant, tack-free, have a pleasant mouth feel and have a pleasant mouth refreshment.

### Example 10

### Comparative Example

1000 g of the dispersion (I) obtained from Example 1 were mixed with 9 g of Loxanol® K12P (III) and 15 g of Stokal STA (III), 30 g of a 0.2% strength aqueous solution of Na saccharin (II) and 30 g of L-menthol Freeflow PN 600129 (II) and then foamed by introducing air using a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm and then dried in a convection oven at 130° C. for 30 minutes.

The aromatized chewing foams prepared in this way were not bite-resistant.

### Example 11

### Comparative Example

1000 g of the dispersion (I) obtained from Example 1 were mixed with 10 g of Plantacare® 1200 UP (III) and 15 g of Stokal STA (III), 30 g of a 0.2% strength aqueous solution of Na saccharin (II) and 30 g of L-menthol Freeflow PN 600129 (II) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the coating material prepared in Example 2 using a brush and then dried in a convection oven at 130° C. for 10 minutes.

The aromatized chewing foams prepared in this way were not bite-resistant.

### Example 12

### Comparative Example

1000 g of the dispersion (I) obtained from Example 1 were mixed with 9 g of Loxanol® K12P (III) and 15 g of Stokal STA (III) and then foamed by introducing air with the help of a hand-mixing device to a foam litre weight of 300 g/l. 40 g of the foamed composition were then poured into a mould made of release paper (VEZ mat, Sappi, Brussels, Belgium) with dimensions 70×140×10 mm (width×depth×height), where a wet layer thickness of 10 mm was achieved. 14 such casting moulds were then dried in an experimental microwave installation (MWT k/1.2-3 LK reg. from EL-A Verfahrenstechnologie Heidelberg, DE) for 30 min at 30% power (3.6 kW at maximum power).

The material was then cut into cubes measuring 10×10×10 mm. All sides of the cubes were painted with the polyurethane dispersion from Example 1 using a brush and then dried in a convection oven at 130° C. for 30 minutes.

The chewing foams prepared in this way have no (long-lasting) mouth refreshment.

1. A process for preparing aromatized chewing foams, comprising
i) formulating one or more polyurethane-polyurea dispersions (I) with foam auxiliaries (III), optionally with thickeners (IV), and optionally with cosmetic additives (V);
ii) foaming the formulation of i);
iii) applying the foamed formulation of ii) to a substrate;
iv) drying the foamed formulation applied to the substrate of iii);
v) shaping the substrate of iv);
vi) refining the surface of the substrate of v);
with the proviso that aromatizing compositions (II) are added in at least one of i) to vi).

2. The process of claim 1, wherein said polyurethane-polyurea dispersions (T) obtained by
A) preparing isocyanate-functional prepolymers of
a1) aliphatic or cycloaliphatic polyisocyanates;
a2) polymeric polyols with number-average molecular weights of from 400 to 8000 g/mol and OH functionalities of from 1.5 to 6;
a3) optionally hydroxy-functional, ionic or potentially ionic and/or nonionic hydrophilizing agents;
B) completely or partially reacting the free NCO groups of said isocyanate-functional prepolymers with
b1) amino-functional compounds with molecular weights of from 32 to 400 g/mol; and/or
b2) amino-functional, ionic, or potentially ionic hydrophilizing agents;
with chain extension, and dispersing the prepolymers in water before, during or after B), wherein any potentially ionic groups present may be converted to the ionic form by partial or complete reaction with a neutralizing agent.

3. The process of claim 2, wherein a1) is selected from the group consisting of 1,6-hexamethylene diisocyanate, isophorone diisocyanate, the isomeric bis(4,4′-iso-cyanatocyclohexyl)methanes, or mixtures thereof, and a2) is at least 70% by weight of a mixture of polycarbonate polyols and polytetramethylene glycol polyols, based on the total weight of the components a2).

4. The process of claim 1, wherein said aromatizing compositions (II) comprise sensorily effective substances which are volatile and are perceptible orthonasally and/or retronasally (aroma substances), or are nonvolatile and are perceptible through interaction with the taste receptors of the human tongue (taste substances).

5. The process of claim 4, wherein said aroma substances comprise a combination of refreshing and cooling active ingredients.

6. The process of claim 4, wherein said taste substances comprise sugar substitutes, sweeteners and/or substances which have a pungent taste, stimulate the flow of saliva in the mouth, cause a feeling of heat and/or a tingling feeling on the skin or on the mucosa.

7. The process of claim 4, wherein said sensorily effective substances are incorporated into a matrix as carrier substance.

8. The process of claim 7, wherein foam auxiliaries (III), thickeners (IV) and cosmetic additives (V) are formulated with said one or more polyurethane-polyurea dispersions.

9. The process of claim 8, wherein said foam auxiliaries are selected from the group consisting of sodium lauryl sulphate, alkyl polyglycoside sulphosuccinamides, ammonium stearate, or mixtures thereof.

10. The process of claim 1, wherein no cariogenic substances during the preparation of said aromatized chewing foams and said aromatized chewing foams do not exceed the critical value of pH 5.7 when carrying out an in vivo plaque pH test.

11. The process of claim 1, wherein the drying in iv) is achieved with microwave radiation at a power of from 250 to 6000 W per kilogram of the foam to be dried.

12. The process of claim 11, wherein the drying in iv) is achieved with conventional thermal drying in addition to said microwave radiation.

13. The process of claim 1, wherein an aromatizing composition (II) is added in i) and further comprising applying a further aromatizing composition (II) as coating (VII) in vi) to the surface of the ready-shaped and dried chewing foam and subsequently drying said coating (VII).

14. The process of claim 13, further comprising applying a coating (VI) after iv) and before applying the aroma coating (VII).

15. An aromatized chewing foam obtained by the process of claim 1.
